(19) United States
(12) Patent Application Publication
(10) Pub. No.: US 2022/0409050 A1
(43) Pub. Date: Dec. 29, 2022

(54) CLINICAL MONITORING OF PATIENT DATA

(71) Applicant: OPUM TECHNOLOGIES LIMITED, Grafton, Auckland (NZ)

(72) Inventor: Andrew John MCDAID, New Lynn, Auckland (NZ)

(73) Assignee: OPUM TECHNOLOGIES LIMITED, Grafton, Auckland (NZ)

(21) Appl. No.: 17/781,303

(22) PCT Filed: Nov. 26, 2020
(86) PCT No.: PCT/NZ2020/050156

§ 371 Date: May 31, 2022

(30) Foreign Application Priority Data
Nov. 29, 2019 (NZ) 759654

(51) Int. Cl. A61B 5/00; G16H 40/67

(52) U.S. Cl. CPC A61B 5/0002; G16H 40/67

(57) ABSTRACT

The invention generally relates to clinical monitoring of patient data. Embodiments provide a system comprising a processor for performing a method and/or computer-implemented method. In certain embodiments the method determines an intervention recommendation for a patient and may comprise analysing patient need data and intervention data to determine the intervention recommendation. In other embodiments, the method monitors, and/or facilitates monitoring of, the monitoring of a patient. The method may comprise receiving providing patient data to a monitor via a user interface on a computing device, monitoring activity and/or one or more actions of the monitor on the user interface, analysing monitoring data, and effecting an action based on the analysis of the monitoring data and/or generating a notification suggesting an action and sending the notification to the monitor based on the analysis of the monitoring data.

### FIELD OF INVENTION

The field of the invention generally relates to clinical monitoring of patient data. More particularly embodiments of the invention relate to systems and/or methods for determining an intervention for a patient, and for monitoring, and/or facilitating monitoring of, the monitoring of a patient.

### BACKGROUND TO THE INVENTION

Monitoring of medical conditions is conventionally conducted through regular visits to a clinician. The clinician then determines what, if any, treatment or further action is required for the condition.

The drawbacks with this arrangement include:
1. It may be time-consuming for both the patient and the clinician to meet face-to-face at a clinic;
2. The need to meet a clinician in person may be expensive, either directly for the patient or for the healthcare system;
3. It may be difficult for some patients to travel to the location of the clinician;
4. The need to schedule time to meet a busy clinician may mean that consultations are not conducted at a time that is optimal, or even particularly useful, for the monitoring or treatment of the condition;
5. The patient may not communicate information to the clinician accurately; and
6. The clinician may not accurately record the information communicated to them by the patient.

Modern technology allows clinicians to monitor patients remotely, for example via phone, email, computer systems and/or video conference, which helps to address some of the above drawbacks. Nevertheless, the need for accurate information to be conveyed by the patient, and to be recorded by the clinician, may not be adequately addressed by the use of such technology. Furthermore, data that is communicated to a clinician remotely, for example through a computer system, may be difficult and/or time-consuming for a clinician to interpret. It may, for example, be difficult to objectively demonstrate that any medical intervention is necessary, and which intervention should be used.

Information on clinical monitoring may be important for insurance purposes. For example, an insurer may want to have confidence that a particular treatment is suitable for a particular patient in order to fund the costs of that treatment. Furthermore, insurers may want information on the level of monitoring being performed on patients in order to determine the level of reimbursement and the need for continuing treatment.

### OBJECT OF THE INVENTION

It is an object of the invention to provide an improved system and/or computer-implemented method for patient monitoring.

Alternatively, it is an object to provide an improved system and/or computer-implemented method for determining, or assisting to determine, an intervention for a patient.

Alternatively, it is an object to provide an improved system and/or computer-implemented method for monitoring, and/or facilitating monitoring of, the monitoring of a patient.

Alternatively, it is an object of the invention to at least provide the public with a useful choice.

### SUMMARY OF THE INVENTION

Aspects of the present invention are directed towards providing a system and/or computer-implemented method used in patient monitoring. The system and/or computer-implemented method may allow the patient to be monitored remotely.

Aspects of the present invention are directed towards providing a system and/or computer-implemented method for determining, or assisting to determine, an intervention for a patient.

According to one aspect of the invention, there is provided a system for determining an intervention recommendation for a patient, the system comprising a processor configured to perform a method comprising:
receiving patient need data representative of at least one need of the patient;
receiving intervention data representative of one or more characteristics of one or more interventions;
analysing the patient need data and the intervention data to determine the intervention recommendation.

According to another aspect of the invention, there is provided a computer-implemented method for determining an intervention for a patient, the method comprising:
receiving patient need data representative of at least one need of the patient;
receiving intervention data representative of one or more characteristics of one or more interventions;
analysing the patient need data and the intervention data to determine the intervention.

In examples the method further comprises receiving patient state data representative of a state of the patient, and analysing the patient state data with the patient need data and the intervention data to determine the intervention recommendation.

In examples the intervention data comprises intervention capability data and/or intervention capacity data.

In examples, the method further comprises:
outputting the intervention recommendation;
receiving the patient need data, patient state data and/or the intervention data from one or more user devices and/or from one or more data stores;
sending survey application data to a user device, the survey application data being configured to be run on the user device in the form of a survey application to collect information from the patient on the patient's needs and/or the patient's present state, the survey application being configured to cause the user device to generate the patient need data and/or the patient state data from the information collected from the user; comparing the patient need data with the intervention data, for example comparing the patient need data with the intervention capability data and/or comparing the patient need data with the intervention characteristic data;
comparing the patient state data with the intervention data, for example comparing the patient state data with the intervention capability data and/or comparing the patient state data with the intervention characteristic data;
for each of the one or more interventions, comparing one or more data elements of the patient need data and/or patient state data with one or more corresponding data elements of the intervention data for the respective intervention and determining a suitability parameter for each of the compared data elements;
for each of the one or more interventions, combining the determined suitability parameters to generate a total recommendation factor;
comparing the total recommendation factors of each of the one or more interventions to determine the intervention recommendation;
determining the total recommendation factor to be zero for an intervention of the one or more interventions if the intervention is categorised in a group that does not correspond to an intervention group determined as being suitable for addressing the need of the patient;
determining the total recommendation factor to be zero for an intervention of the one or more interventions if the intervention data for the intervention comprises a contra-indication that corresponds to one of the data elements of the patient state data.
for each of the one or more interventions, determining an intervention dose quantity for addressing each of the at least one need of the patient;
comparing the patient need data with the patient state data to determine gap data representative of one or more gaps between the at least one need of the patient and the state of the patient, and analysing the gap data to determine the intervention recommendation; and
comparing the gap data to the intervention capacity data to determine an intervention capacity recommendation, wherein the intervention recommendation comprises the intervention capacity recommendation.

In examples, the system further comprises:
one or more wearable devices configured to generate patient state data and send the patient state data to the processor;
one or more user devices configured to generate patient need data, patient state data and/or intervention data, and to send the patient need data, patient state data and/or intervention data to the processor; and
one or more display devices configured to display the intervention recommendation.

Further aspects of the invention are directed towards providing a system and/or computer-implemented method for monitoring a patient. Further aspects of the invention are directed towards providing a system and/or computer-implemented method for monitoring, and/or facilitating monitoring, of the monitoring of a patient.

According to another aspect of the invention, there is provided a system for monitoring, and/or facilitating monitoring of, the monitoring of a patient, the system comprising a processor configured to perform a method comprising:
receiving patient data;
processing the patient data;
providing the patient data and/or information representative of the patient data to a monitor via a user interface on a computing device;
monitoring activity and/or one or more actions of the monitor on the user interface to generate monitoring data representative of the monitoring activity and/or action(s) of the monitor on the user interface;
analysing the monitoring data; and
effecting an action based on the analysis of the monitoring data and/or generating a notification suggesting an action and sending the notification to the monitor based on the analysis of the monitoring data.

According to another aspect of the invention, there is provided a computer-implemented method for monitoring, and/or facilitating monitoring of, the monitoring of a patient, the method comprising:
receiving patient data;
processing the patient data;
providing the patient data and/or information representative of the patient data to a monitor via a user interface on a computing device;
monitoring activity and/or one or more actions of the monitor on the user interface to generate monitoring data representative of the monitoring activity and/or action(s) of the monitor on the user interface;
analysing the monitoring data; and
effecting an action based on the analysis of the monitoring data and/or generating a notification suggesting an action and sending the notification to the monitor based on the analysis of the monitoring data.

In examples:
The patient data is representative of any one or more aspects of the patient's health, physical state, physiological state, mental state and/or physical activity performed by the patient. The patient data may be data representative of one or more physical parameters, for example blood pressure, oxygen saturation levels, body temperature, heart rate, height and/or weight. The patient data may also or alternatively be representative of physical activity performed by the patient, for example number of steps, range-of-motion, strength, spatiotemporal gait parameters, posture (e.g. time in posture), activity/exercise (time and quality of), or data generated by one or more wearable devices worn by the patient; and
The step of processing the patient data comprises any one or more of: storing the patient data in a memory, categorising the patient data, aggregating the patient data; and analysing the patient data.

In examples, the method further comprises receiving input from the monitor to effect an action.

In examples, the system further comprises:
one or more wearable devices configured to generate patient data and send the patient data to the processor; and
one or more display devices configured to display the patient data and/or information representative of the patient data.

Further aspects of the invention, which should be considered in all its novel aspects, will become apparent to those skilled in the art upon reading of the following description which provides at least one example of a practical application of the invention.

### BRIEF DESCRIPTION OF THE DRAWINGS

One or more embodiments of the invention will be described below by way of example only, and without intending to be limiting, with reference to the following drawings, in which:

FIG. 1 is a schematic illustration of a patient monitoring system according to one embodiment of the technology;

FIG. 2 is a schematic diagram of data flow in an exemplary medical intervention determination and method according to an embodiment of the technology;

FIG. 3 is an illustration of a smartphone displaying a screen on a user interface prompting a patient to provide information according to one embodiment of the technology;

FIG. 4 is an illustration of the smartphone of FIG. 3 displaying a screen on the user interface according to an embodiment of the technology;

FIGS. 5-7 are illustrations of the smartphone of FIG. 3 displaying screens on a user interface prompting a patient to provide information according to one embodiment of the technology;

FIG. 8 is a flow chart illustration of an exemplary method of determining an intervention according to certain embodiments of the technology;

FIG. 9 is an exemplary method of monitoring the monitoring of a patient according to certain forms of the technology;

FIG. 10 is an illustration of a smartphone displaying a screen on a smartphone according to one embodiment of the technology;

FIG. 11 is an illustration of the smartphone of FIG. 10 displaying a screen according to an embodiment of the technology;

FIG. 12 is an illustration of the smartphone of FIG. 10 displaying a screen making a suggestion to the HCP according to an embodiment of the technology;

FIG. 13 is an illustration of the smartphone of FIG. 10 displaying a screen making another suggestion to the HCP according to an embodiment of the technology;

FIG. 14 is an illustration of the smartphone of FIG. 10 displaying a screen making another suggestion to the HCP according to an embodiment of the technology.

### DETAILED DESCRIPTION OF PREFERRED EMBODIMENTS OF THE INVENTION

Exemplary Patient Monitoring System

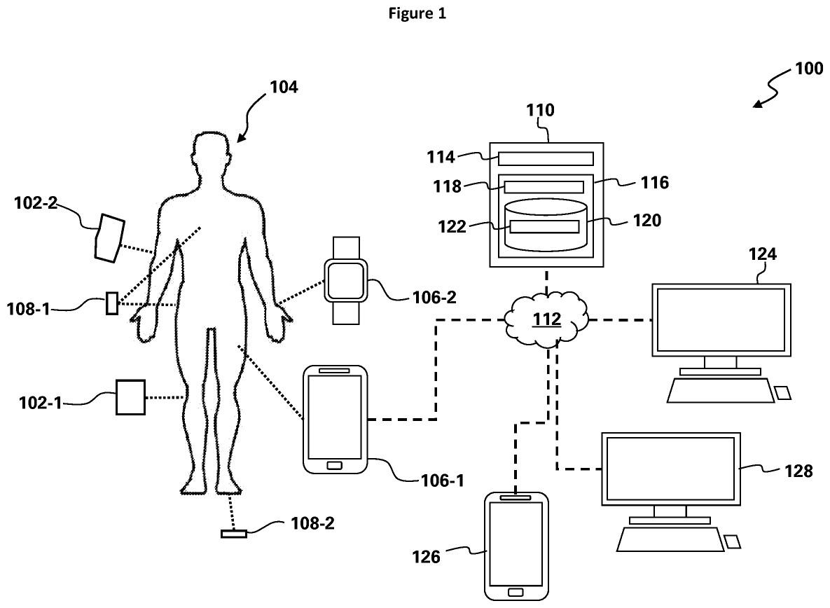

FIG. 1 is a schematic illustration of a patient monitoring system 100 according to certain embodiments of the technology. Patient monitoring system 100 is used in certain forms of the technology to monitor patients according to certain methods that will be described in more detail later.

Patient monitoring system 100 includes one or more wearable devices 102 (for example, knee orthosis 102-1 and/or elbow orthosis 102-2) configured to be mounted to a corresponding body part(s) of a body of a patient 104 in use. In certain forms the wearable devices 102 may take the form of those described in PCT Application No. PCT/NZ2018/050085, the contents of which are incorporated herein by reference.

The system 100 further includes one or more reference sensors, for example comprised as part of intelligent user devices 106 (for example, smart phone 106-1 and/or smart watch 106-2) and/or dedicated reference sensor devices 108 (for example, an inertial measurement unit (IMU) 108-1 and/or smart insole 108-2). Intelligent user devices 106 may be considered to be a type of wearable device.

In exemplary embodiments, data from one or more of the wearable devices 102, user devices 106, and/or reference sensor devices 108 may be communicated to a remote processing service 110 via a network 112 (for example a cellular network, or another network optionally comprising various configurations and protocols including the Internet, intranets, virtual private networks, wide area networks, local networks, private networks using communication protocols proprietary to one or more companies—whether wired or wireless, or a combination thereof). For example, the smart phone 106-1 may operate an application capable of interfacing with the remote processing service 110 over the Internet.

The patient monitoring system 100 may also comprise other user devices through which one or more users may interface with the remote processing service 110. For example, the patient monitoring system 100 may comprise patient computing device 124, clinician computing device 128 and clinician smart phone 126, each of which is configured to send information to, and receive information from, the remote processing service 110.

Among other functions, the remote processing service 110 may record data, perform analysis on the received data, and report to one or more user devices. In this exemplary embodiment, the remote processing service 110 is illustrated as being implemented in a server—for example one or more dedicated server devices, or a cloud-based server architecture. By way of example, cloud servers implementing the remote processing service 110 may have processing facilities represented by one or more processors 114, memory 116, and other components typically present in such computing environments. In the exemplary embodiment illustrated the memory 116 stores information accessible by processors 114, the information including instructions 118 that may be executed by the processors 114 and data 120 that may be retrieved, manipulated or stored by the processors 114. The memory 116 may be of any suitable means known in the art, capable of storing information in a manner accessible by the processors, including a computer-readable medium, including a non-transitory computer-readable medium, or other medium that stores data that may be read with the aid of an electronic device. The processors 114 may be any suitable processing device known to a person skilled in the art. Although the processors 114 and memory 116 are illustrated as being within a single unit, it should be appreciated that this is not intended to be limiting, and that the functionality of each as herein described may be performed by multiple processors and memories, that may or may not be remote from each other.

The instructions 118 may include any set of instructions suitable for execution by the processors 114. For example, the instructions 118 may be stored as computer code on the computer-readable medium. The instructions may be stored in any suitable computer language or format. Data 120 may be retrieved, stored or modified by processors 114 in accordance with the instructions 118. The data 120 may also be formatted in any suitable computer readable format. Again, while the data is illustrated as being contained at a single location, it should be appreciated that this is not intended to be limiting—the data may be stored in multiple memories or locations. The data 120 may include databases 122 storing data such as historical data associated with one or more of the one or more of the wearable devices 102, user devices 106, and/or reference sensor devices 108, and the results of analysis of same.

It should be appreciated that in exemplary embodiments the functionality of the remote processing service 110 may be realised in a local application (for example, on smart phone 106-1 or 126, or another personal computing device 124 or 128), or a combination of local and remote applications. Further, it should be appreciated that data may be transferred from one or more of the devices by other means—for example wired communication links, or transfer of storage devices such as memory cards.

The results of analysis, and/or underlying data, may be displayed on any suitable display device—for example smart phone 106-1 or 126, or computing device 124 or 128.

Determining a Medical Intervention

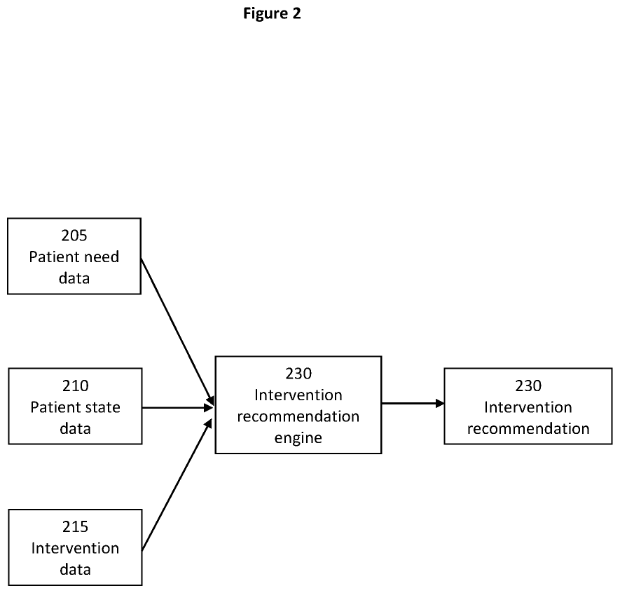

In certain forms of the present invention the patient monitoring system 100 operates as a medical intervention determination system and implements a computer-implemented method for determining or recommending an intervention for a patient. FIG. 2 is a schematic diagram of data flow in an exemplary medical intervention determination and method.

Patient Need Data

A first type of data that may be used by the medical intervention determination system according to some embodiments is patient need data 205. Patient need data 205 is data that is representative of at least one need that a patient has, for example a medical and/or physiological capability. Typically, the patient need data 205 may be representative of a capability that the patient wants but does not currently have, and can therefore be considered to be an unmet patient need.

Non-limiting examples of needs represented by patient need data 205 are being able to:
Move a specified distance and/or for a specified amount of time, for example walk 2 km, walk up a flight of stairs, run for 10 minutes, or perform more than 5000 steps per day;
Move or lift a weight a specified distance, for a specified amount of time, and/or a specified number of times with a specified part of the body, for example lift a 10 kg weight with the right forearm or repeatedly push a 20 kg weight with leg extensions;
Move a joint through a specified angle, with or without resistance;
Perform a specified action, for example any of the previous examples, swing a golf club or throw a ball;
Use a bodily function, for example talk, hear, see, urinate;
Meet a certain physiological or physical state, for example, have a specified weight, body temperature and/or oxygen saturation level; and
Meet a certain mental state, for example pain or confidence level.

Patient need data 205 may be provided by a patient, a clinician and/or from a data store, for example a patient's medical records. For example, in certain embodiments, patient need data 205 may be generated by a user device, for example patient smartphone 106-1, patient personal computing device 124, clinician smartphone 126 or clinician personal computing device 128, in response to patient 104 or a clinician entering information into an application running on the user device. The patient need data 205 may be sent by the user device to the remote processing service 110 via network 112.

The user device may be configured to run one or more applications that prompt a patient or clinician to provide information that may be used to generate the patient need data 205. The application may be a dedicated application installed locally on the user device or may be a generic application, such as a web browser. The application may receive survey application data from the remote processing service 110 and be configured to cause the user device to generate the patient need data 205 from the information collected from the user. The application may present one or more questions, for example in the form of a survey or a questionnaire to the user to prompt information to be provided. The remote processing service 110, or an application on a user's device, may be configured to prompt a user (e.g. a patient or clinician) to provide patient need data 205 on a regular basis, for example daily. Alternatively, the user may be provided with the means to provide information in an unprompted manner.

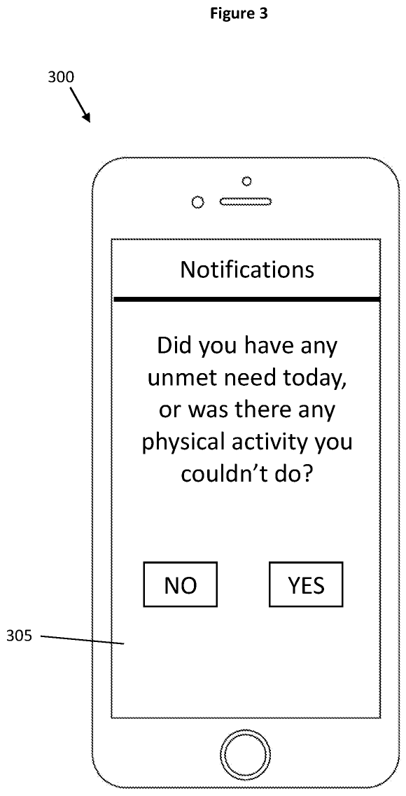

FIG. 3 is an illustration of a smartphone 300 displaying a screen 305 on a user interface prompting a patient to provide information according to one embodiment of the invention. Screen 305 comprises a question asking the patient whether there was any physical activity they could not do today, and provides a mechanism by which the patient can answer the question. In the example of FIG. 3 this mechanism is displaying two virtual buttons on the touchscreen display by which the patient can submit answers to the question.

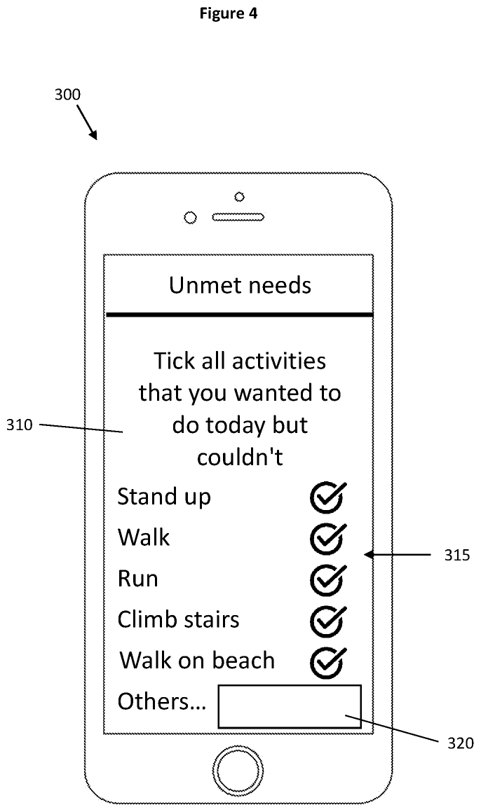

FIG. 4 is an illustration of smartphone 300 displaying a screen 310 on the user interface that may be presented to the patient if they respond to the question “did you have any unmet need today, or was there any physical activity you couldn't do?” shown in FIG. 3 to indicate that there was physical activity they could not do today, according to an embodiment of the invention. Screen 310 comprises a list of options of activities that the patient may not have been able to complete and prompts the user to select the applicable activities. The user may input the applicable activities by checking tick boxes 315 or inputting text into a text field 320, for example. In other embodiments the smartphone 300, or other user device, may be configured to display a plurality of screens to a user in order to prompt information from the user on the needs or unmet needs of the patient. The displayed screens may comprise a hierarchy of screens prompting increasingly detailed information, for example first prompting for detail on an activity that could not be achieved and then prompting for specific information about that activity, for example the length of a walk that could not be achieved or the height of stairs that could not be climbed. The information collected by smartphone 300 may be used to generate patient need data 205. The generation of the patient need data 205 from the information inputted by the user may occur in the user smartphone 300 (or other user device), in the remote processing service 110 or another location.

Examples of questions that may be presented to a patient in other examples are: “did you have to stop walking today because you were too fatigued?”, “were you not able to participate in an activity because you couldn't walk on the terrain?” and “did you want to climb stairs today but couldn't?”

Patient State Data

A second type of data used by the medical intervention determination system in some embodiments is patient state data 210. Patient state data 210 is data that is representative of a state of a patient. In certain forms of the invention the patient state data 210 is representative of a present state of the patient. In alternative forms the patient state data 210 may be additionally or alternatively representative of a past state of the patient. The patient state data 201 may relate to, or be indicative of, a medical, physiological, functional, biomechanical and/or mental state of a patient. Additionally or alternatively, the patient state data 210 may be personal data or historical medical/physiological/mental state data.

Non-limiting examples of patient states represented by patient state data 201 are:
Patient's personal information, e.g. name, age, address, email address, telephone number, income;
Patient's medical information, e.g. height, weight, body mass index (BMI), blood type, body temperature, oxygen saturation level, fall risk, a result of any medical test;
Patient's prior and/or existing medical conditions, prior and/or existing treatments, prior and/or existing prescriptions; and
Patient's current performance data in performing specified actions, for example the patient being capable of performing a certain number of press ups, distance walked, kilograms lifted, and/or gait parameters;
Patient's current physical or mental state, including symptoms, e.g. range-of-motion (ROM), pain level, confidence level, isometric or dynamic strength of a joint against a power or passively resistive exoskeleton, joint stiffness;
Patient's sleep state, e.g. data representative of the quantity and/or quality of sleep; and
Patient medical images, e.g. X-rays, CT, MRI images (or data representing such images).

Patient state data 210 may be provided by a patient, a clinician and/or from a data store, for example a patient's medical records. For example, in certain embodiments, patient state data 210 may be generated by a user device, for example patient smartphone 106-1, patient personal computing device 124, clinician smartphone 126 or clinician personal computing device 128, in response to patient 104 or a clinician entering information into an application running on the user device. The patient state data 210 may be sent by the user device to the remote processing service 110 via network 112.

The user device may be configured to run one or more applications that prompt a patient or clinician to provide information that may be used to generate the patient state data 210. The application may be a dedicated application installed locally on the user device or may be a generic application, such as a web browser. The application may receive survey application data from the remote processing service 110 and be configured to cause the user device to generate the patient state data 210 from the information collected from the user. The application may present one or more questions, for example in the form of a survey or a questionnaire to the user to prompt information to be provided. The remote processing service 110, or an application on a user's device, may be configured to prompt a user (e.g. a patient or clinician) to provide patient state data 210 on a regular basis, for example daily. Alternatively, the user may be provided with the means to provide information in an unprompted manner.

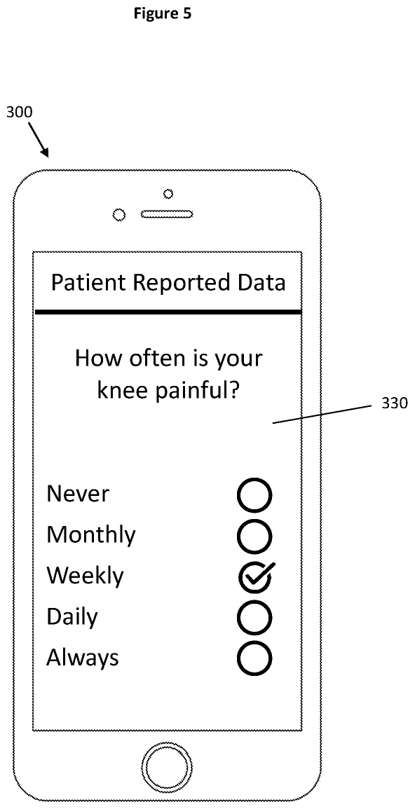

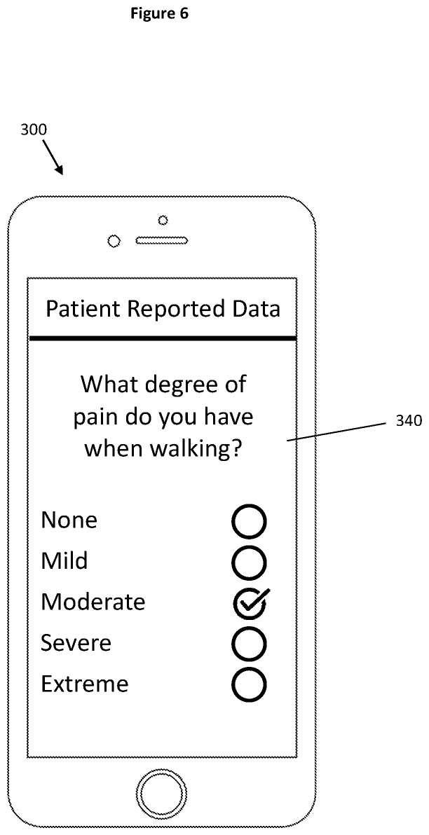

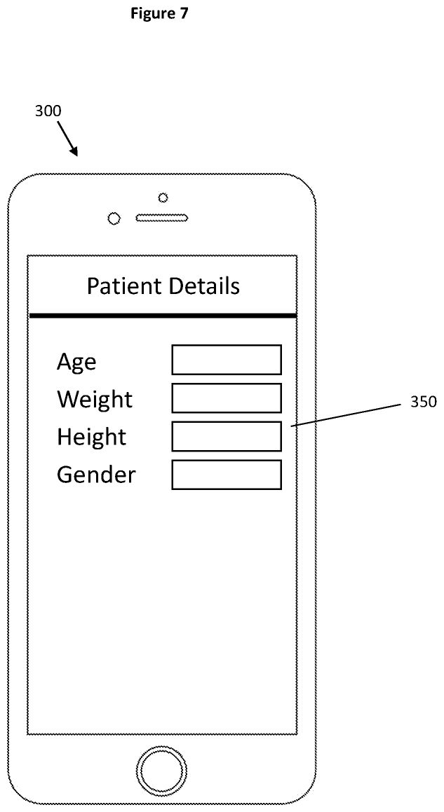

FIGS. 5, 6 and 7 are illustrations of a smartphone 300 displaying screens 330, 340 and 350 respectively on a user interface prompting a patient to provide information according to one embodiment of the invention. Screens 330, 340 and 350 prompt for information that may be used to generate patient state data 210 through various means, for example questions with tick box answers, questions with free text answers, questions with multiple-choice answers, etc. The smartphone 300 may be configured to display a hierarchy of screens to increasingly obtain more detailed information on the state of the patient, for example firstly prompting the patient to identify the type of a physiological condition suffered, then prompting the patient to indicate the frequency, severity and/or duration of the condition.

The information on the patient state collected by smartphone 300 may be used to generate patient state data 210. The generation of the patient state data 210 from the information inputted by the user may occur in the user smartphone 300 (or other user device), in the remote processing service 110 or another location.

Additionally or alternatively, patient state data may be generated and/or provided by one or more wearable devices 102. For example a wearable device 102 may be configured to monitor, and generate data indicative of, a physiological parameter of a patient, for example the patient's heart rate, temperature, glucose levels, knee laxity, angle between joints, number of steps walked per day, walking speed, activities (e.g. walking up stairs, squats, lungs), a patient's ability to perform those activities (e.g. speed of walking upstairs (for example measured in number of steps per second), depth of squat, isometric strength against a powered or passive resistance exoskeleton, and to send said data to the remote processing service 110. Examples of wearable devices 102 are knee orthoses/exoskeleton 102-1, elbow orthosis/exoskeleton 102-2, smart watch 106-2, IMU 108-1 and smart insole 108-2. In certain forms the wearable devices 102 may take the form of those described in PCT Application No. PCT/NZ2018/050085, the contents of which are incorporated herein by reference.

The table below shows examples of patient state data 210 for three exemplary patients. In some forms, the patient state data 210 is stored in a database in relational form, e.g. in a table. The data may be sparse, i.e. some data fields may be unpopulated.

| Patient State | Patient A | | Patient B | | Patient C | |
|---|---|---|---|---|---|---|
| Age | 20 | | 67 | | 54 | |
| Height | 200 | cm | 180 | cm | — | |
| Weight | 120 | kg | 80 | kg | 75 | kg |
| Gender | — | | F | | M | |
| Pregnant? | NO | | YES | | NO | |
| Walking ability | 5,022 | | 1,400 | | 9,001 | |
| (average steps per day) | | | | | | |
| Knee ROM | — | | 50° | | 20° | |
| Running speed | 1.5 | m/s | 5 | m/s | 2 | m/s |

| | | | |
|---|---|---|---|
| Smoker? | NO | YES | YES |
| Diabetes? | YES | — | NO |
| Pain when walking | None | Mild | Severe |
| Motivation | High | Low | High |

Intervention Data

A third type of data that may be used by the medical intervention determination system in some embodiments is intervention data 215. Intervention data 215 is data that is representative of a property, characteristic and/or quality of an intervention. The intervention may be a treatment or rehabilitation device, for example. Non-limiting examples of interventions represented by intervention data 215 are:
Drugs, for example a course of drugs or a specified quantity of a drug or its active ingredient;
Joint braces, for example knee, elbow or ankle braces;
Movement aids, for example prosthetics, walking sticks, zimmer frames;
Implants, hearing aids, spectacles;
Surgical interventions, for example knee replacements, ligament repairs/reconstructions;
Therapeutic interventions, for example physiotherapy, occupational therapy, speech/language therapy, massage, dry needling;
Respiratory interventions, for example continuous positive airway pressure (CPAP);
Psychological treatments; and/or
Exercise regimes.

Interventions may be classed in groups. Groups of interventions may be related by being different interventions of the same type, for example exemplary groups of interventions may be: exercise programs; assistive devices; drugs; and therapy programs. Groups of interventions may further be categorised into sub-groups for example sub-groups of exercise programs may be exercise programs for specific parts of the body, and sub-groups of assistance devices may be knee braces, and walking sticks. Intervention data 215 may indicate which group and/or sub-group each intervention is part of.

Some interventions may be mutually exclusive, for example some drugs may not be able to be taken in combination, or only one type of anti-inflammatory should be prescribed at any one time, or the patient can only have one type of knee brace or prosthetic. In certain examples, interventions in the same group and/or sub-group may be mutually exclusive. Intervention data 215 for a given intervention may include identifiers for other interventions that are mutually exclusive to the given intervention. This may be used to indicate that where an intervention recommendation is made for the given intervention (according to the process described below), an intervention recommendation should not also be made for an intervention that is mutually exclusive with the given intervention.

Intervention data 215 may comprise intervention capability data. Intervention capability data is data that is representative of the capability of an intervention. A capability of the intervention may be the type, nature and/or severity of a condition that can be treated by the intervention.

Non-limiting examples of intervention capabilities represented by intervention capability data are:
Medical conditions that can be treated by the intervention, and parameters indicative of their type/severity;
Claims/evidence of efficacy of intervention, for example quantity and/or quality of clinical evidence, level of efficacy (e.g. strong evidence that a knee brace will reduce the likelihood of a patient re-injuring their knee after ACL surgery by 10%, or low evidence that a specific drug will increase activity levels by 50%);
Characteristics of patients that are suitable for treatment by the intervention, for example demographic information (age, gender, ethnicity), physical characteristics (height, weight, blood type); and/or
Contraindications associated with the intervention, e.g. pregnancy, use of pacemaker, etc.

Intervention data 215 may additionally or alternatively comprise intervention characteristic data. The intervention characteristic data may be representative of any characteristic of an intervention, for example any of the interventions listed above. The characteristics of an intervention may include the amount, duration, intensity, dosage, type, make/model, size or any other characteristic of an intervention. The intervention data 215 may be in the form of parameters indicative of any of these characteristics. It will be appreciated that the possible characteristics are dependent on the type and nature of the intervention.

Intervention data 215 may be provided by a clinician, an intervention supplier, an intervention manufacturer, an intervention service provider, a patient and/or a data store, for example data records of any party having information on an intervention.

The table below shows examples of intervention data 215 for three exemplary interventions. In some forms, the intervention data 215 is stored in a database in relational form, e.g. in a table. The data may be sparse, i.e. some data fields may be unpopulated. Intervention data 215 may be stored in a structure and format that facilitates a comparison with the patient state data 210 and/or the patient need data 205, for example quantitative parameters are stored in the same units and qualitative parameters are stored in the same or similar formats (e.g. binary values are YES and NO or ON and OFF or 0 and 1, same list of selectable options).

| Intervention | | | | | |
|---|---|---|---|---|---|
| data | Intervention 1 | | Intervention 2 | | Intervention 3 |
| Contraindication | <4 | years old | >80 | years old | none |
| Age | | | | | |
| Contraindication | YES | | YES | | NO |
| Pregnancy? | | | | | |
| Contraindication | <50 | kg | <50 | kg | >120 kg |
| weight | | | | | |

| | | | |
|---|---|---|---|
| Suitable | Arthritis | Back pain | Amputation |
| conditions | | | |
| Claims | Decrease pain | Increase range | Improve |
| | increase | of movement. | walking |
| | walking speed | Increase ability | speed |
| | Increase ROM | to play golf | |
| | | Increase strength | |

Intervention Determination Engine

In embodiments of the invention, one or more of the different types of data, i.e. patient need data 215, patient state data 210 and/or intervention data 215, is provided to an intervention determination engine 220. Intervention determination engine 220 is a functional part of embodiments of the invention that is configured to analyse the different types of data input into it and to generate an intervention recommendation 230. The intervention determination engine 220 will be understood to be implemented in certain embodiments of the invention by a processing system such as remote processing service 110. That is, the analysis performed by the intervention determination engine 220 is achieved through implementing the processing capabilities of the remote processing service 110.

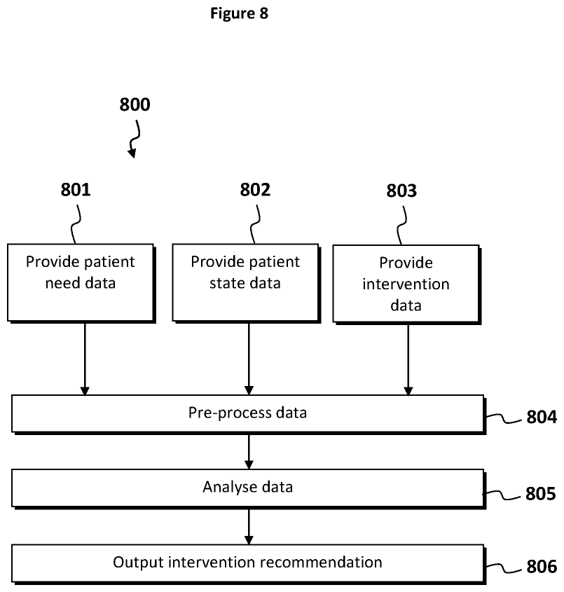

FIG. 8 is a flow chart illustration of an exemplary method 800 of determining an intervention according to certain embodiments of the invention.

At step 801, patient need data 205 is provided to the remote processing service 110. The patient need data 205 may be provided by any suitable means, including being received from user devices as discussed above. In alternative forms of the technology, the patient need data 205 is pre-provided to the remote processing service 110 and is stored as data 120, for example in databases 122.

At step 802, patient state data 210 is provided to the remote processing service 110. The patient state data 210 may be provided by any suitable means, including being received from user devices and/or wearable devices as discussed above. In alternative forms of the technology, the patient state data 210 is pre-provided to the remote processing service 110 and is stored as data 120, for example in databases 122.

At step 803, intervention data 215 is provided to the remote processing service 110. The intervention data 215 may be provided by any suitable means, including as discussed above. In alternative forms of the technology, the intervention data 215 is pre-provided to the remote processing service 110 and is stored as data 120, for example in databases 122.

In certain forms of the technology method 800 may comprise a step 804 of pre-processing any one or more of the patient need data 205, patient state data 210 and intervention data 215 in order to transform the data into a form that is suitable for subsequent processing steps, for example to convert inputted data into a standardised form that can be compared by remote processing service 110 to other data of the same or similar nature. For example, quantitative data may be converted into the same units and qualitative data may be mapped to one of a plurality of pre-set options for each data type. The need for data pre-processing step 804 may be dependent on the source of the data. For example, patient state data 210 obtained from electronic medical records maintained by one health provider may already be in a suitable form due to data entry requirements set by that health provider, while patient state data 210 obtained from the electronic medical records of another health provider may be in a different form and require pre-processing to map that data to a form that is similar to the data from the first health provider. Similarly, some patient need data 205 and intervention data 215 obtained from different sources from other of the respective type of data may need pre-processing to convert that data into a form that is processable in the same way as the other type of data. Intervention data 215 may be provided to remote processing service 110 through an interface (e.g. a user interface and/or an API) that requires the data to be provided in certain formats, where the interface is configured such that those formats are already suited for analysis by the remote processing service 110 to avoid pre-processing. Certain forms of the technology are not limited by the form of data used.

At step 805 the remote processing service 110 analyses the patient need data 205, the intervention data 215 and, optionally, the patient state data 210 to determine the intervention recommendation 230. More detail on step 805 is provided below.

At step 806 the remote processing service 110 outputs the intervention recommendation 230. The intervention recommendation 230 may be output in any one or more of a number of ways including, but not limited to:
Displaying the intervention recommendation 230 on a display of a device, for example smart phone 106-1, patient computing device 124, clinician computing device 128 and/or clinician smart phone 126;
Sending the intervention recommendation 230 to another device or processing system;
Storing the intervention recommendation 230 in a data store; and
Generating an order or prescription for an intervention, for example a rehabilitation device or drug.

It will be appreciated that method 800 for determining an intervention may be performed multiple times for each patient. The intervention recommendation may differ each time based on changes in the state of the patient, the patient's needs and/or the nature of available interventions. That is, a first intervention determination may recommend a first intervention, for example the first intervention for a patient with a knee injury may be an exercise plan. Then, at a later time, a second intervention determination may recommend a second intervention. The second intervention may differ from the first intervention, for example as a result of patient recovery or other change to the data under analysis. In the example of the patient with a knee injury, after the patient performs the prescribed exercise plan for a period of time, they may lose weight and be able to walk a certain distance. As a result of these changes in the patient's state, the intervention determination may change to the use of a microprocessor knee for walking upstairs, or along the beach.

Analysis to Determine the Intervention Recommendation

It has already been described that, at step 805, the remote processing service 110 analyses the patient need data 205, patient state data 210 and, optionally, the intervention data 215 to determine the intervention recommendation 230. It will be appreciated that, in the context of FIG. 2, the intervention determination engine 220 performs this analysis. There is now described more detail on the analysis process. It will be appreciated that, where method steps are described in the ensuing description, these steps are carried out by the remote processing service 110/intervention determination engine 220.

The remote processing service 110 compares the patient need data 205 with the intervention data 215 to determine what one or more interventions meet the patient's one or more needs. If a characteristic of an intervention is identified as meeting, or being close to meeting, a need of a patient then the remote processing service 110 determines that the intervention is suitable for the patient and the intervention recommendation 230 is based on that intervention.

More particularly, the remote processing service 110 may compare intervention capability data with the patient need data 205 to determine what one or more interventions meet the patient's one or more needs. Additionally or alternatively, the remote processing service 110 may compare intervention characteristic data with the patient need data 205 to determine what one or more interventions meet the patient's one or more needs.

The remote processing service 110 may also analyse the patient state data 210 in its analysis. It may compare the patient state data 210 with the intervention data 215 to determine whether an intervention is suitable for meeting a patient's needs. The intervention data 215 compared in this analysis may be intervention capability data and/or intervention characteristic data. For example, the remote processing service 110 may compare demographic details of the patient with intervention capability data in the form of demographic data indicating the characteristics of patients for which an intervention is suitable and, depending on the results of that comparison, determine that the intervention is suitable or is not suitable for meeting the patient's need(s).

In one example, there is a patient John Smith. The patient state data 210 representative of John's state includes:
name: John Smith;
age: 35;
medical condition: amputee with right knee and lower leg missing;
prosthetic used: knee prosthetic model ‘ABC’;
daily activity rate: medium, 1000 steps per day;
fall risk: low.

The patient need data 205 representative of John's needs may include:
wants to walk up stairs.

The intervention data 215 may include data representative of an intervention ‘microprocessor-controlled knee model XYZ’, which data includes:
prosthetic type: microprocessor-controlled knee;
claims: can help amputees walk, can help amputees walk up stairs;
indication: above knee amputee;
suitable demographic: 15-60 years old;
contra-indications: low activity (less than 400 steps per day, high fall risk).

The intervention data 215 may further comprise intervention capacity data indicative of the amount, duration, intensity, etc of the intervention required to meet certain needs, for example in the case of microprocessor-controlled knee model XYZ this may take the form ‘wear permanently for helping amputees walk’, ‘wear when awake for helping amputees walk further’ and/or ‘wear during physical activity for helping amputees climb up stairs’.

The remote processing service 110 compares the corresponding elements of the data to determine the applicability of the intervention (as well as other candidate interventions) to the patient and their situation. For example, one comparison is made between the intervention capability data in the form of the suitable demographic data and the corresponding patient state data, in this case the patient's age. In the given example, since the patient's age sits within the range that the intervention is suitable for, a favourable comparison is made. Similarly other patient state data is compared to the intervention data, for example the daily activity rate data is compared to the activity indication in the contra-indications to determine whether the intervention is suitable. Furthermore, patient need data may be compared to the intervention data, for example the patient wanting to walk up stairs is compared to the claim data that microprocessor-controlled knee model XYZ can help patients walk up stairs. Furthermore, patient need data may be compared to the intervention capacity data to determine the amount, duration, intensity, etc of the intervention required by the patient. For example, the need to wear microprocessor-controlled knee model XYZ during physical activity may be identified for assisting John Smith to walk upstairs.

Having made these comparisons a determination is made as to whether this intervention, and a plurality of other candidate interventions, is/are suitable for the patient and an intervention recommendation is made accordingly.

In some forms of the technology, the remote processing service compares one or more data elements of the patient need data and/or patient state data with one or more corresponding data elements of the intervention data and determines a suitability parameter for each comparison between data elements made, for example a binary yes/no suitability parameter or a suitability parameter indicative of a degree of suitability. For each intervention analysed, the determined parameters are combined, for example, summed or other function of the parameters, in order to generate a total recommendation factor. The total recommendation factors of all interventions analysed may be compared to determine the intervention recommendation 230, for example the intervention with the highest total recommendation factor is the recommended intervention.

In some forms, if a binary suitability parameter for any intervention is ‘no’, or if a suitability parameter is below a predetermined threshold, the remote processing service 110 may disregard that intervention from the determination of the intervention recommendation 230. For example, if a characteristic of the patient or their condition as given in the patient state data is inconsistent with, or is counter to, a contra-indication of an intervention in the intervention data, then that intervention is ruled out from consideration. For example, the suitability parameter is set to zero as a result of the contra-indication.

In some forms, for any execution of the analysis step 805, all interventions are assessed to determine suitability parameters and total recommendation factors for all interventions, and the remote processing service is configured to select the intervention with the highest total recommendation factor as the recommended intervention. Alternatively, the remote processing service may be configured to output the a predetermined (e.g. selectable) number of possible interventions, being those with the highest total recommendation factors.

In some forms, the remote processing service calculates the suitability parameters and total recommendation factors for a sub-set of the interventions, where the sub-set is a group and/or a sub-group of interventions selected based on certain criteria. For example, the patient need data 205 may comprise category data, which categorises the patient's need according to the general type of intervention suitable for addressing it, e.g. physiotherapeutic intervention, pharmaceutical intervention, etc. The remote processing service then performs the analysis to determine the recommended intervention(s) only within the groups and/or sub-groups of interventions suitably categorised. This may reduce the processing requirement of the analysis step.

In some forms, the remote processing service is configured to determine an intervention dose quantity for every intervention analysed in the analysis step 805. The intervention dose quantity is the amount of the intervention determined as being necessary to meet the patient's need. The intervention dose quantity may be determined based on the patient need data 205, the patient state data 210 and/or the intervention data 215, for example the intervention capacity data.

The remote processing service may be configured to determine the intervention dose quantity for all interventions in groups and/or sub-groups of interventions not matching the patient's need as zero, and also all interventions that have a contra-indication matching some aspect of the patient's state data as zero. For example, the patient need data 205 may indicate a requirement to run a certain distance without pain, e.g. 20 km. This type of patient need data 205 may be categorised as being suitable for a physiotherapeutic intervention, in which case the intervention dose quantities for all non-physiotherapeutic interventions, e.g. pharmaceutical interventions is set to zero.

The remote processing service may be configured to determine the intervention dose quantity for all interventions in groups and/or sub-groups of interventions that match the patient's need according to a predetermined determination method. The method may be, for example, a rule-based algorithm, look-up table, parameterised model, trained data-driven algorithm or trained machine learning system. In the example of rule-based algorithms, each physiotherapeutic intervention may be associated with a dose quantity calculation algorithm, which is executed to determine the ‘dose’ of each intervention (i.e. the quantity of the intervention to be undertaken). For example, the number of squats (which may be considered the ‘dose’ of the intervention that is performing squats) may be determined by a formula such as: Number of squats=0.05*(100−<patient age>)*<desired distance to run in km>+10. Other formulae for the dose of the intervention may be determined for any physiotherapeutic intervention categorised as being suitable for assisting with the patient need. In this case, the intervention recommendation 230 may be a plurality of interventions and the determined intervention dose quantity for each of the plurality of interventions.

In certain forms of the technology, the analysis step 805 may comprise performing a gap analysis comparing the patient state data 210 with the patient need data 205 to determine gap data representative of one or more gaps between a patient's state and the patient's needs. In these forms, the remote processing service 110 may also analyse the gap data when determining the intervention recommendation 230, for example by comparing the gap data with the intervention data. For example, a patient's patient need data 205 may indicate that the patient has an unmet need of walking 10 km. The patient's patient state data 210 may indicate that the patient is only capable of walking 1 km. From these data it may be determined that gap data representative of a gap between the patient's unmet need and their state (in this case their present state) is the need to be able to walk an additional 9 km.

The table below illustrated examples of gap data corresponding to types of patient state data 210 and patient need data 205. For quantitative data types as in the examples shown, the gap data may be the numerical difference between the patient state data 210 and the corresponding patient need data 205.

| | Current Ability | Desired ability | |
|---|---|---|---|
| | (Patient | (Patient | Unmet need |
| Activity | state data) | need data) | (Gap data) |
| Distance walking | 1 km | 10 km | 9 km |
| without pain | | | |
| Squats in 30 sec | 10 | 15 | 5 |
| Stairs climbing speed | 20/min | 20/min | 0 |
| Knee range of motion | 25° | 90° | 65° |
| Play golf | 9 holes | 18 holes | 9 holes |

The remote processing service 110 may compare this gap data with stored intervention data 215 to identify one or more interventions that are suitable for enabling the patient to walk an additional 9 km.

Other data may also be taken into account in performing this analysis, for example patient state data 205 indicating the nature of the patient's incapacity. For example, in the case of a patient with a knee problem (indicated by the patient state data 205), the remote processing service 110 may determine that the intervention suitable to enable the patient to walk an additional 9 km is a particular make/model of knee brace or a specified exercise regime.

Furthermore, the gap data may be compared to intervention capacity data for each intervention to determine an intervention capacity recommendation, which is comprised as part of the intervention recommendation 230. For example, if the gap data indicates the patient needs to walk an additional 9 km, a comparison with the intervention capacity data for a knee brace may result in the intervention recommendation including an indication of the need to wear the knee brace throughout the day, or when walking. Alternatively a comparison with the intervention capacity data for an exercise regime may result in the intervention recommendation including an indication to perform specified muscles stretches 10 times per day every day for three weeks.

In certain forms of the technology, the analysis of the data in step 805 is performed by one or more predetermined algorithms executed by remote processing service 110. In alternative forms of the technology, the analysis is performed by remote processing service 110 configured to comprise an artificial intelligence, for example an artificial neural network (ANN). The ANN may be trained with exemplary data and intervention recommendations in order to re-configure itself through machine learning processes.

Intervention Recommendation

As previously described, at step 806, an intervention recommendation 230 is output. Intervention recommendation 230 may be considered to be data representative of a recommendation of an intervention that is suitable to be output in any of the ways described above.

In certain forms, the intervention recommendation 230 comprises data representative of a unique identifier of a selected intervention, for example an identification number and/or the name (e.g. make/model) of an intervention. Examples may be ‘microprocessor-controlled knee model XYZ’, ‘drug no. 123456’ or ‘exercise regime A123Z’.

In other forms, the intervention recommendation 230 may comprise a plurality of suggestions for interventions and may further comprise a plurality of supplementary data, each supplementary data associated with one of the interventions and being representative of the degree of suitability of an intervention to a patient, or any additional information associated with the recommendation. For example, one form of intervention recommendation 230 may be: ‘knee brace ABC—recommendation score 4.8; knee brace XYZ—recommendation score 4.7’. Another form may be: ‘elbow brace ABC—recommended provided patient has no prior history of lateral epicondylitis’.

As explained above, in certain forms, the intervention recommendation 230 may comprise a plurality of interventions and the determined intervention dose quantity for each of the plurality of interventions.

The intervention recommendation 230 may be output to a user, for example a clinician, by a suitable output mechanism, for example by being output by a clinician computing device 128 and/or clinician smart phone 126.

The remote processing service 110 may be configured to receive as an input an intervention selected by a clinician. For example, a clinician may be able to select an icon on a display interface indicating the intervention out of a plurality of recommended interventions they deem to be the most suitable to meet the needs of a particular patient. The remote processing service 110 may be configured to input data representative of the selected intervention into the intervention determination engine 220, which may be configured to learn from this selection. For example, the selected intervention may be input into as training data into a neural network forming part of the intervention determination engine 220.

Monitoring of Patient Monitoring

In certain forms of the technology there is provided systems and methods for monitoring, and/or facilitating monitoring, of the monitoring of a patient. In some forms of the invention the remote processing service 110 of patient monitoring system 100 performs the monitoring of the patient, while in other forms the patient monitoring system 100 enables, and assists, a human person, for example a clinician or other healthcare professional (HCP), to perform the monitoring of the patient. The entity performing the monitoring, whether it be remote processing service 110 or a human using patient monitoring system 100 may be referred to as a monitor.

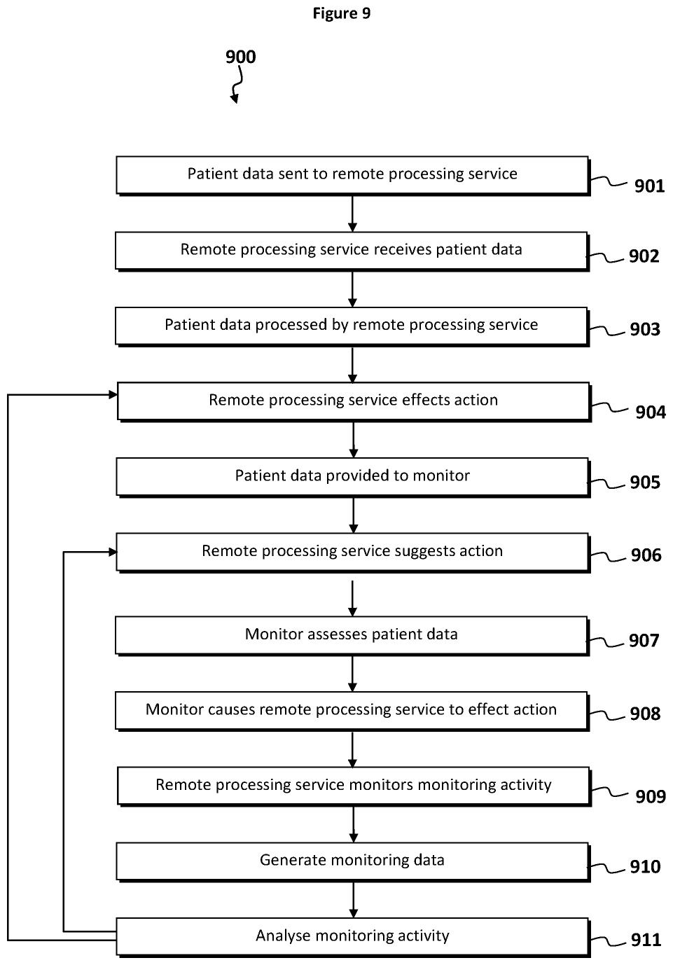

FIG. 9 is an exemplary method 900 of monitoring the monitoring of a patient 104 according to certain forms of the technology. The steps of method 900 will be described with reference to patient monitoring system 100, which will be understood to be configured to perform, or assist with performing, various steps of the method 900. The ordering of the steps of method 900 illustrated in FIG. 9 and explained below is for explanatory purposes only, and is non-limiting to forms of the invention. In other forms, the steps may be carried out in another order.

At step 901, patient data is sent to the remote processing service 110, for example over network 112. The patient data may be data representative of any aspect of a patient's health, physical state, physiological state, mental state and/or physical activity performed by the patient. The patient data may be data representative of one or more physical parameters, for example blood pressure, oxygen saturation levels, body temperature, heart rate, height and/or weight. The patient data may also or alternatively be representative of physical activity performed by the patient, for example number of steps, range-of-motion, strength, spatiotemporal gait parameters, posture (e.g. time in posture), activity/exercise (time and quality of), or data generated by one or more wearable devices 102 worn by a patient. For example a wearable device 102 may be configured to monitor, and generate data indicative of, a physiological parameter of a patient, for example the patient's heart rate, temperature, glucose levels, knee laxity, angle between joints, etc, and to send said data to the remote processing service 110. Examples of wearable devices 102 are knee orthoses/exoskeleton 102-1, elbow orthosis/exoskeleton 102-2, smart watch 106-2, IMU 108-1 and smart insole 108-2.

At step 902 the patient data is received by the remote processing service 110.

At step 903 the patient data is processed by the remote processing service 110. Processing of the data by one or more processors 114 of the remote processing service 110 may include any one or more of: storing the data in memory 116, categorising the data, aggregating the data and analysing the data. In the ensuing description, any reference to “patient data” will be understood to refer to the patient data in the form it is received by the remote processing service 110 and/or data produced during the data processing step 903. This may include data representative of information derived from the patient data, for example data summary information in the form of graphs, tables, charts or the like.

The nature of the analysis performed on the data depends on the nature of the data and what the remote processing service 110 is configured to analyse. In certain forms of the technology, analysis of the patient data by the remote processing service 110 may determine that a parameter or quantity indicative of a patient's physical or physiological state satisfies predetermined criteria, for example is above a threshold, below a threshold and/or outside a specified range. The parameter may be biometric data, physiological data and/or data generated by a wearable device. As described with reference to the next step of method 900, the remote processing service 110 may be configured to perform an action if the parameter or quantity satisfies the predetermined criteria.

At step 904 the remote processing service 110 may effect an action. The action may be effected as the result of analysis performed on the patient data. Step 904 may occur before, at the same time as, or after, the later steps of method 900. The action may be to contact, for example to send a message or email to the patient 104, the monitor and/or another party. Another example of an action is to schedule a consultation, for example a video call, appointment or other type of meeting, between the patient 104 and the healthcare professional. In certain forms, patient data analysed by the remote processing service 110 may be indicative of a patient's level of compliance with a prescribed treatment plan. A prescribed treatment plan may be, for example, a programme of drugs, or an exercise regime. If the patient's level of compliance is outside predetermined criteria defined (for example by the monitor) as being adequate, then an action may be performed. The type of action may depend on the level of compliance, and the severity of the action may increase the lower the compliance. For example the remote processing service 110 may be configured to perform a first type of action (for example sending the patient 104 a notification) if the level of compliance is below a first threshold, and to perform a second type of action (for example notifying a healthcare professional) if the level of compliance is below a second threshold, where the second threshold is lower than the first threshold and where the second type of action is considered more severe (i.e. more of an intervention/treatment correction) than the first type of action. Further actions of differing levels of severity may be performed for other levels of compliance.

At step 905 the patient data is provided to the monitor via a user interface on a computing device. In forms of the technology where the remote processing service 110 acts as the monitor, this step will be understood to be fulfilled by the step 901 of sending the patient data to the remote processing service 110. In other forms of the technology, the data and/or derived information is provided to a monitor in the form of a clinician or HCP, for example by sending the data/information to a clinician computing device 128 and/or a clinician smart phone 126, and/or displaying the data/information to the HCP on a display device of a clinician computing device 128 and/or a clinician smart phone 126. As mentioned earlier, it will be understood that what is provided to the monitor may be the patient data in the form it is received by the remote processing service 110, but in other forms it is data representative of information derived from the patient data, for example data summary information in the form of graphs, tables, charts or the like.

At step 906 the remote processing service 110 may optionally generate a notification suggesting an action and sending the notification to the monitor. The suggested action may be something the monitor should perform or otherwise. The remote processing service 110 may generate an action suggestion based on analysis of monitoring activity and/or action(s) performed by the monitor at an earlier time, for example in a similar or analogous circumstance. The determination of the action suggestion that the remote processing service 110 generates is described further with reference to step 911 below. The suggestions may be provided to the patient 104 and/or healthcare professional through any communication means, for example text, call, email and/or through a healthcare app on a user computing device. Non-limiting examples of suggestions include suggestions to:
Schedule a meeting with a healthcare professional;
Remember to follow a treatment plan;
Perform specific exercises;
Seek advice from another healthcare professional (for example with a different medical speciality);
Increase/decrease the number of repetitions in an exercise and/or the frequency at which an exercise is performed;
Advance/regress to a difference phase of exercise/therapy plan;
Advance/regress to a different intervention; and
End an intervention and/or monitoring.

At step 907 the monitor assesses the patient data. For example, the healthcare professional or clinician reads and assesses the patient data and/or data representative of information derived from the patient data that is displayed to them on a computing or other device. The monitor may use a user interface of the computing or other device to interact with the display of the data to them to access the information they want to see, or to manipulate the data in any way to obtain the information they are interested in. For example, the healthcare professional may select a particular parameter from a drop-down list and subsequently be shown data for the patient relating to that parameter.

At step 908 the monitor causes the remote processing service 110 to effect an action. The action may be an action effected by the remote processing service 110 as the result of input and/or a trigger provided by the monitor having assessed the patient data. In examples, a healthcare professional, having reviewed patient data, may determine that a patient's treatment plan requires changing. The healthcare professional therefore inputs the new treatment plan into their computing device, this new plan is sent as data to the remote processing service 110, this results in the details of the treatment stored by remote processing service 110 to alter, and this is communicated to the patient through a message being sent to the patient's smartphone 106-1, or the data indicative of the prescribed treatment plan that is presented to the patient through an app accessed through the smartphone 106-1 being altered. In another example, the action may be to send the patient a reminder to do something, for example to take a prescribed medicine, do an exercise or visit a health practitioner.

It will be understood that step 908 is an optional step in that the monitor may cause the remote processing service 110 to effect no action. This may be, for example, because the monitor is happy with the patient data they have viewed.

At step 909 the remote processing service 110 monitors the monitoring activity and/or action(s) of the monitor where the monitor interacts with the patient monitoring system 100 via the user interface on the computing device on which the patient data is provided. Non-limiting examples of the actions of the monitor that may be monitored in this step are:
How much time the monitor spends viewing patient data;
What patient data the monitor views and/or how long the monitor spends viewing which patient data, for example how much time the monitor spends viewing certain web pages of a web-based portal, where the nature of the patient data displayed on each web page is known (this may be determined by determining the duration that the respective web pages are displayed on a display of, for example, the clinician computing device 128 and/or the clinician smart phone 126);
How the monitor interacts with the patient data, for example by registering mouse movements, mouse clicks and/or direction of the monitor's gaze on a screen displaying the patient data by eye-tracking;
How much time has elapsed since the monitor last viewed patient data, and/or last viewed particular types of patient data;
What actions the monitor causes the remote processing service 110 to effect, for example by registering selections of action buttons displayed on a screen; and
The nature and/or details of the patient data when the monitor causes the remote processing service 110 to effect those actions.

The skilled addressee will appreciate how a conventional computer use monitoring system may be used by the remote processing service 110 to perform step 909. In certain examples, the remote processing service 110 may commence a timer on initiating display of patient data to a monitor, for example displaying a web page or application page on clinician computing device 128 and/or clinician smart phone 126, and cease the timer once the page is no longer displayed or after a predetermined time of non-activity on the page, for example 20 seconds of non-activity (the assumption being that the monitor would have ceased looking at the data after a certain amount of time of non-activity on the page). The time-of-viewing data from the timer is saved in memory 116, along with data identifying the nature of the patient data being viewed on the page, in relation to the identification of the patient whose data is being monitored and in relation to the identification of the monitor performing the monitoring. In another example, the remote processing service 110 may record any selection actions (e.g. mouse clicks) occurring when patient data is displayed in memory 116, the selection action data identifying the data being displayed at the time, what actions were selected and the identification of the patient and/or monitor performing the monitoring. In some cases the selection action may be the selection of a button that causes additional data to be displayed, for example more specific data on a particular condition, patient history data, and the like.

At step 910 the remote processing service 110 may generate monitoring data representative of the monitor's monitoring action(s) through the patient monitoring system 100 on the user interface. The monitoring data may comprise, for example, data representative of any one or more of the actions listed above. The remote processing service 110 may further display the monitoring data through a user interface and/or generate a report detailing, illustrating and/or summarising the monitoring data.

At step 911 the remote processing service 110 analyses the monitoring activity and/or action(s) of the monitor by analysing the monitoring data. In certain forms of the technology the remote processing service 110 is configured to perform machine learning in order to determine one or more actions to effect at step 904 and/or to suggest at step 906. This action/these actions are then effected and/or suggested, as the case may be, at the appropriate time in the future. In order to perform this step, remote processing service 110 may be configured to comprise an artificial intelligence, for example an artificial neural network (ANN). The ANN may be trained with exemplary data of monitoring activity and/or action(s) of a monitor and corresponding actions to effect and/or actions to suggest to a monitor in order to re-configure itself through machine learning processes.

In certain forms, monitoring data obtained from previous uses of the patient monitoring system 100 (i.e. previous monitoring of monitoring activity) may be used as training data to train the remote processing service 110 how to suggest actions. It will be appreciated that such training data may be used as an input to, for example an ANN in order to re-configure itself.

In one example, monitoring data obtained through prior use of the patient monitoring system 100 may indicate that monitors usually initiate the system 100 to send the patient a reminder to do physiotherapeutic exercises after three days of non-compliance. If such monitoring data is used to train the remote processing service 100, the ANN may be re-configured so that, in future, if three days of non-compliance for such exercises for any patient is detected, the remote processing service 100 generates a suggestion to the monitor (for example through a pop-up window or notification, for example sent via email, SMS or through a dashboard) to send a reminder to the patient to perform their exercises. The remote processing service 100 may also record how the monitor responds to such a suggestion, e.g. whether they agree with the suggestion and perform an action to cause the remote processing service 100 to send a reminder to the patient, or whether they decline to act upon the suggestion. Data indicative of this response is further used as training data to train the remote processing service 100.

Monitoring of Patient Monitoring—Examples

There will now be described some examples of the method 900 of monitoring the monitoring of a patient 104 according to certain forms of the technology.

In one example, a patient 104 wears a knee brace. The knee brace may take the form of the knee brace described in PCT Application No. PCT/NZ2018/050085, the contents of which are incorporated herein by reference. The knee brace is configured to sense a number of parameters, for example the movement of the knee brace over time, the forces exerted by the patient on the knee brace over time, and the number of bends exerted by the patient when wearing the knee brace in a certain period of time (e.g. a day). The knee brace is further configured to transmit data representative of the sensed parameters to the remote processing service 110. The remote processing service 110 stores the data from the knee brace in memory 116.

The remote processing service 110 analyses the data received from the knee brace. In doing so, it is determined that the movement of the knee brace over time (which is indicative of the patient's range-of-motion (ROM) of the knee and may therefore be termed ROM data) fulfils one or more particular criteria. The criteria may be indicative of unplanned recovery, for example outside two standard deviations of the population normal ROM at the same time since a surgical procedure. In this case, the remote processing service 110 may be configured to automatically create a video call event and to send meeting requests for the video call event to the monitor and the patient 104. If the ROM data is indicative of recovery within a particular range (for example inside two standard deviations of the population normal ROM at the same time since a surgical procedure), the remote processing service 110 may be configured to take no action.

In another example, the remote processing service 110 may calculate a parameter indicative of the patient's compliance with treatment. For example, in the case of a patient wearing a knee brace, a clinician may have prescribed to the patient a certain amount of physical activity, for example a number of knee bends to perform, or number of kilometres to walk, per day. The remote processing service 110 may be configured to analyse the data from the knee brace and determine whether the patient is complying with treatment, for example to calculate a compliance parameter. The remote processing service 110 may be configured to take predetermined steps dependent on the level of compliance calculated. For example, if the level of compliance is above 100%, no action is taken, or the patient may be sent a congratulatory message. The remote processing service 110 may be configured to take further actions dependent on the level of compliance, for example: if the level of compliance is below another predetermined value, for example 80%, the remote processing service 110 may be configured to send the patient a notification reminding them to perform their prescribed treatment plan; if the level of compliance is below 50% a phone call between the patient and clinician is scheduled; and if the level of compliance is below 20% a visit from the clinician to the patient is scheduled.

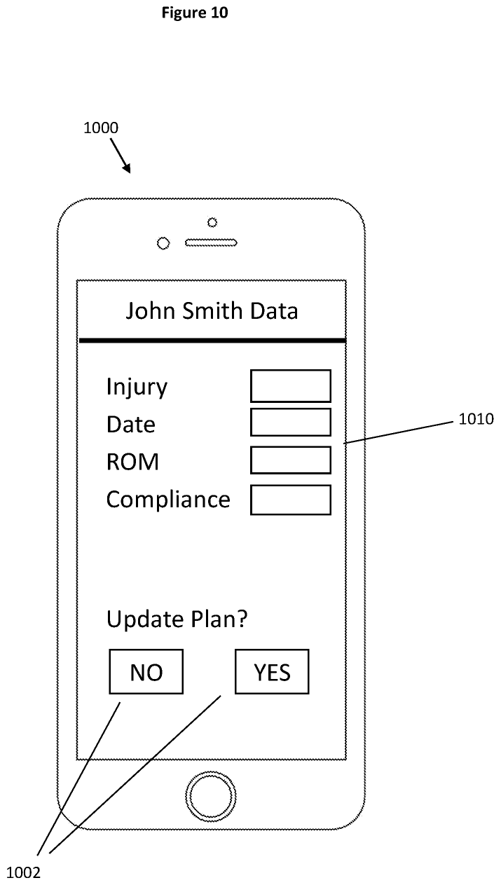

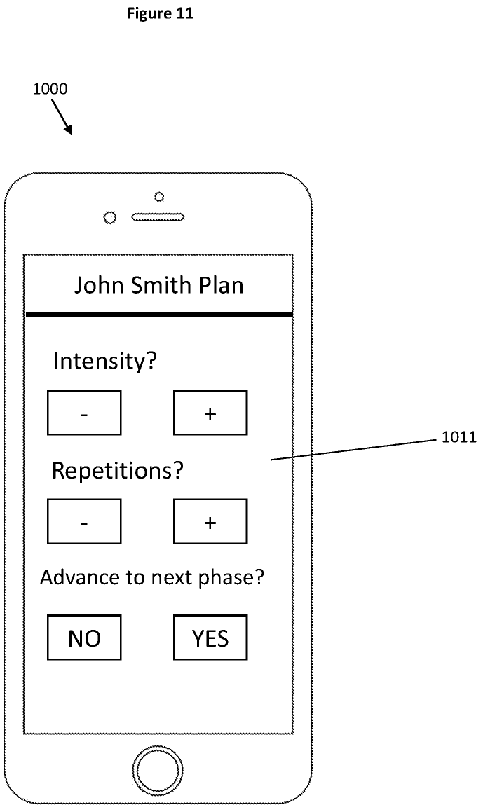

In this example, remote processing service 110 is configured to generate a display of information representative of the data received from the knee brace on a healthcare professional (HCP) device. For example, the remote processing service 110 may generate a graph of the angle of the knee brace over time, for example over the course of a day, and displays said graph to the HCP on a clinician computing device 128 at the end of every day. In another example, data is displayed to the HCP in the manner shown in FIG. 10. FIG. 10 is an illustration of a smartphone 1000 displaying a screen 1010 on a smartphone according to one embodiment of the invention. Screen 1010 displays information indicative of a patient's activity associated with the knee brace that day, for example the ROM and a parameter indicative of compliance. The screen 1010 also provides a mechanism for the HCP to input any changes to the treatment plan that are required, for example buttons 1002. If the HCP selects that the treatment plan requires changing, the device may be configured to display a screen 1011, such as is shown in FIG. 11, which is an illustration of smartphone 1000 displaying a screen 1011 according to an embodiment of the invention. Screen 1011 may provide the HCP with easy ways to alter the treatment plan, for example by providing buttons enabling the HCP to incrementally increase or decrease the intensity and/or number of repetitions of an exercise, for example knee bends. Once the HCP has altered the treatment plan on their device and clicked ‘save’, the remote processing service 110 is configured to provide the updated treatment plan information to the patient, for example by sending a message to one of the patient's devices.

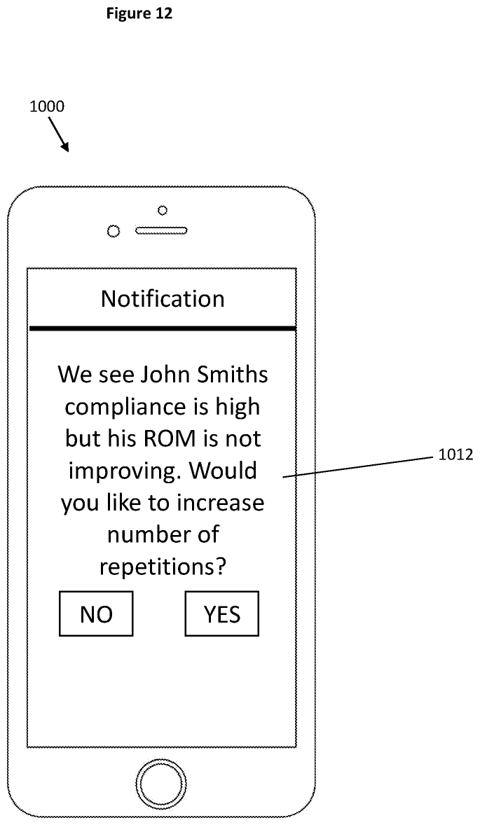

In this example, the remote processing service 110 generates data indicative of the HCP's activity on their smartphone. For example, the remote processing service 110 generates and stores monitor activity data representative of the HCP having viewed screen 1010, the type and value of the parameters displayed on screen 1010, and the actions that the HCP performed as a result of those parameters, for example the incremental changes to the intensity and repetitions on screen 1011. The remote processing service 110 performs analysis of the monitor activity data and determines suggestions for actions at a later date. For example, the remote processing service 110 may record that the number of repetitions is increased when the ROM parameter and the compliance parameter has a certain value, or based on a trend of these parameters over time. If similar values occur again, or dependent on the trend of the values, the remote processing service 110 may generate a notification to the HCP suggesting that similar changes to the number of repetitions be made. FIG. 12 is an illustration of smartphone 1000 displaying a screen 1012 making a suggestion to the HCP according to an embodiment of the invention and giving the HCP an input mechanism to effect the suggestion. Screen 1012 may be shown to the HCP on a later day than the change to the repetitions inputted by the HCP to screen 1011. In the suggestion shown on screen 1012 the HCP is prompted to increase the number of repetitions because the remote processing service 110 has determined a similar situation, or a trend of parameters that has led to an increase in repetitions, similar to what has occurred in the past.

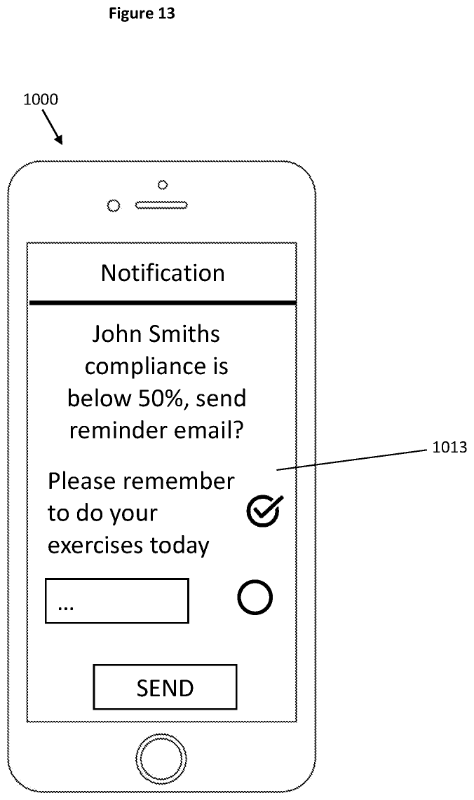

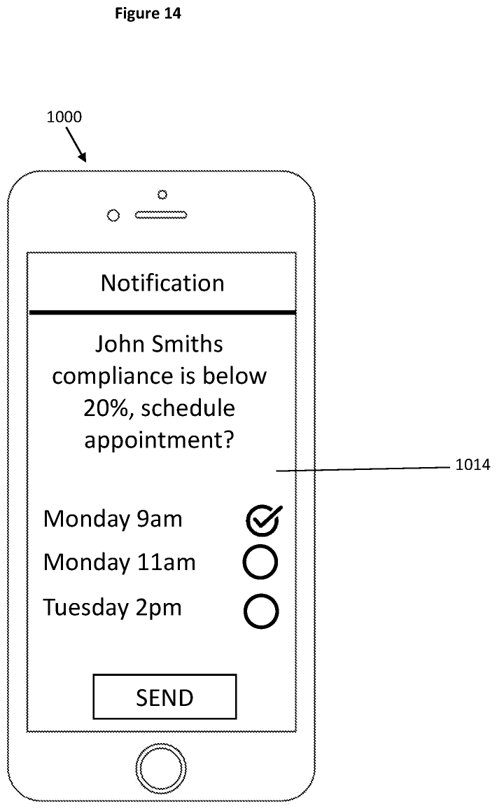

FIG. 13 is an illustration of smartphone 1000 displaying a screen 1013 making another suggestion to the HCP according to an embodiment of the invention. In the suggestion displayed on screen 1012 the HCP is prompted to provide input to cause the remote processing service 110 to send a notification, for example a reminder email, to the patient as a result of the parameter indicative of the patient's level of compliance being below a predetermined value; in this case 50%. The HCP is configured to determine whether to trigger this action to be taken and may be presented with options for wording in the reminder email. Another example is illustrated in FIG. 14, which is an illustration of smartphone 1000 displaying a screen 1014 making another suggestion to the HCP according to an embodiment of the invention. In this example, the remote processing service 110 has determined a compliance parameter being below 20% and prompts the HCP to schedule an appointment as a result. As explained, in other embodiments the remote processing service 110 may be configured to take these steps without first prompting the HCP to confirm whether to take those steps or not. In certain forms, the patient monitoring system 100 may be configurable to enable a party, for example the HCP, to decide what situations result in automatic action by the remote processing service 110 and what situations result in prompts being sent to the HCP.

In another example the remote processing service 110 monitors the amount of patient monitoring performed by a monitor, for example a HCP, over a period of time. If the monitoring time is below a predetermined value, the remote processing service 110 may be configured to effect an action. For example, if the remote processing service 110 determines that a HCP has reviewed a patient's data for less than 20 minutes in a month, the remote processing service 110 may be configured to send the HCP a reminder to review that patient's data in more detail and/or the remote processing service 110 may be configured to schedule a telephone call or an appointment between the HCP and the patient. In some forms of the technology, the remote processing service 110 may be configured to take such steps prior to the end of the monitoring period, for example if it is provided with a rule that a patient's data needs monitoring for 30 minutes in a month and it determines that, with only three days of the month left, there are still 10 minutes of monitoring to be performed, it may be configured to effect certain actions, for example to generate and send reminders and/or appointment requests.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

The invention may also be said broadly to consist in the parts, elements and features referred to or indicated in the specification of the application, individually or collectively, in any or all combinations of two or more of said parts, elements or features.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

1. A system for determining an intervention recommendation for a patient, the system comprising a processor configured to perform a method comprising:
receiving patient need data representative of at least one need of the patient;
receiving intervention data representative of one or more characteristics of one or more interventions; and
analysing the patient need data and the intervention data to determine the intervention recommendation.

2. A system as claimed in claim 1, wherein the method further comprises receiving patient state data representative of a state of the patient, and analysing the patient state data with the patient need data and the intervention data to determine the intervention recommendation.

3. A system as claimed in claim 1, wherein the intervention data comprises intervention capability data and/or intervention capacity data.

4. A system as claimed in claim 1, wherein the method further comprises, for each of the one or more interventions:
comparing one or more data elements of the patient need data and/or patient state data with one or more corresponding data elements of the intervention data for the respective intervention and determining a suitability parameter for each of the compared data elements;
combining the determined suitability parameters to generate a total recommendation factor; and
comparing the total recommendation factors of each of the one or more interventions to determine the intervention recommendation.

5. A system as claimed in claim 4, wherein the method further comprises determining the total recommendation factor to be zero for an intervention of the one or more interventions if the intervention is categorised in a group that does not correspond to an intervention group determined as being suitable for addressing the need of the patient.

6. A system as claimed in claim 5, wherein the method further comprises determining the total recommendation factor to be zero for an intervention of the one or more interventions if the intervention data for the intervention comprises a contra-indication that corresponds to one of the data elements of the patient state data.

7. A system as claimed in claim 1, wherein the method further comprises, for each of the one or more interventions, determining an intervention dose quantity for addressing each of the at least one need of the patient.

8. A system as claimed in claim 1, wherein the method further comprises comparing the patient need data with the patient state data to determine gap data representative of one or more gaps between the at least one need of the patient and the state of the patient, and analysing the gap data to determine the intervention recommendation.

9. A system as claimed in claim 8, wherein the method further comprises comparing the gap data to the intervention capacity data to determine an intervention capacity recommendation, wherein the intervention recommendation comprises the intervention capacity recommendation.

10. A system as claimed in claim 1, wherein the system further comprises one or more wearable devices configured to generate patient state data and send the patient state data to the processor.

11. A system for monitoring, and/or facilitating monitoring of, the monitoring of a patient, the system comprising a processor configured to perform a method comprising:
receiving patient data;
processing the patient data;
providing the patient data and/or information representative of the patient data to a monitor via a user interface on a computing device;
monitoring activity and/or one or more actions of the monitor on the user interface to generate monitoring data representative of the monitoring activity and/or action(s) of the monitor on the user interface;
analysing the monitoring data; and
effecting an action based on the analysis of the monitoring data and/or generating a notification suggesting an action and sending the notification to the monitor based on the analysis of the monitoring data.

12. A system as claimed in claim 11, wherein the step of processing the patient data comprises any one or more of: storing the patient data in a memory; categorising the patient data; aggregating the patient data; and analysing the patient data.

13. A system as claimed in claim 11, wherein the method further comprises receiving input from the monitor to effect an action.

14. A system as claimed in claim 11, wherein the system further comprises one or more wearable devices configured to generate patient data and send the patient data to the processor.

15. A computer-implemented method for monitoring, and/or facilitating monitoring of, the monitoring of a patient, the method comprising:
receiving patient data;
processing the patient data;
providing the patient data and/or information representative of the patient data to a monitor via a user interface on a computing device;
monitoring activity and/or one or more actions of the monitor on the user interface to generate monitoring data representative of the monitoring activity and/or action(s) of the monitor on the user interface;
analysing the monitoring data; and
effecting an action based on the analysis of the monitoring data and/or generating a notification suggesting an action and sending the notification to the monitor based on the analysis of the monitoring data.

16. A system as claimed in claim 15, wherein the step of processing the patient data comprises any one or more of:
storing the patient data in a memory;
categorising the patient data;
aggregating the patient data; and
analysing the patient data.

17. A system as claimed in claim 15, wherein the method further comprises receiving input from the monitor to effect an action.
